(19) United States
(12) Patent Application Publication
(10) Pub. No.: US 2025/0197364 A1
(43) Pub. Date: Jun. 19, 2025

(54) METHOD FOR PRODUCING CIS-4-AMINOTETRAHYRDROFURAN-2-CARBOXYLIC ACID ESTERS

(71) Applicant: BAYER AKTIENGESELLSCHAFT, Leverkusen (DE)

(72) Inventors: Dirk BROHM, Leverkusen (DE); Andreas REMBIAK, Leverkusen (DE); Anton LISHCHYNSKYI, Leverkusen (DE)

(21) Appl. No.: 18/848,735

(22) PCT Filed: Mar. 23, 2023
(86) PCT No.: PCT/EP2023/057452

§ 371 Date: Sep. 19, 2024

(30) Foreign Application Priority Data
Mar. 28, 2022 (EP) 22164857.9

(51) Int. Cl. C07D 307/24

(52) U.S. Cl. CPC C07D 307/24

(57) ABSTRACT

The present invention relates to a novel process for preparing cis-4-aminotetrahydrofuran-2-carboxylic esters of the general formula (I) and salts thereof.

The present invention relates to a novel process for preparing cis-4-aminotetrahydrofuran-2-carboxylic esters of the general formula (I) and salts thereof.

cis-Methyl 4-aminotetrahydrofuran-2-carboxylate hydrochloride (CAS 1304126-28-0) of the general formula (I) (where R¹=Me) is an important building block for the synthesis of crop protection products (WO 2012/130798, WO 2021/170464).

The first published synthesis route is by Walker et al. (Synthesis 2011, 7, 1113-1119). The precursor used is methyl 4-(tert-butoxycarbonylamino)furan-2-carboxylate (CAS 1170719-58-0, Wolter et al., Org. Lett. 2009, 11, 2804). However, methyl 4-(tert-butoxycarbonylamino)furan-2-carboxylate cannot be prepared in an economically viable manner on an industrial scale on account of the high costs of the reagents for the preparation, the low yields, safety risks and phosphate waste. By way of example, this requires a lithiation with sec-butyllithium (34% yield) and a Curtius rearrangement with DPPA (diphenylphosphonic azide). This route is therefore not feasible for the preparation of cis-4-aminotetrahydrofuran-2-carboxylic esters on an industrial scale.

In addition, the synthesis via methyl 4-bromofuran-2-carboxylate (CAS 58235-80-6, WO 2021/170464) is costly on account of the bromination and the subsequent inefficient coupling with tert-butyl carbamate (25% yield, WO 2021/170464) and leads to large amounts of waste.

There is therefore a high demand for novel, cost-effective and environmentally friendly processes for synthesizing cis-4-aminotetrahydrofuran-2-carboxylic esters, whereby the corresponding crop protection products can then also be prepared on an industrial scale.

WO 2012/137982 describes the reductive amination of methyl 4-oxotetrahydrofuran-3-carboxylate with benzylamine. It is possible in this substitution pattern to first dehydrate the ketone with the benzylamine to form an enamine which is stabilized via conjugation to the ester. The subsequent reduction of the double bond was then carried out with sodium triacetoxyborohydride.

This invention describes a novel process for preparing cis-4-aminotetrahydrofuran-2-carboxylic esters proceeding from 4-oxotetrahydrofuran-2-carboxylic esters of the general formula (II). The preparation of this starting substance is described for example in WO 2016/205633.

In light of the prior art described above, the present invention is based on the object of finding a process for preparing the specified compounds which is cost-effective and can be used on an industrial scale. It is also desirable to obtain these compounds in a high yield and in high purity, such that they do not have to be subjected to any further complex purification.

The object described above—simple, cost-effective and industrial-scale preparation—is achieved by a process for preparing compounds of the general formula (I) or salts thereof

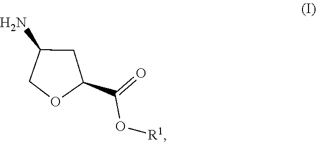

in which
R¹ is (C₁-C₆) alkyl or (C₃-C₆) cycloalkyl,
characterized in that in a first step compounds of the general formula (II)

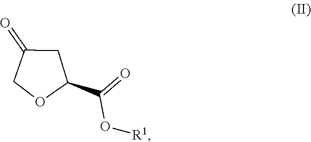

in which
R¹ is as defined above,
react with an amine of the general formula (III) or salts thereof

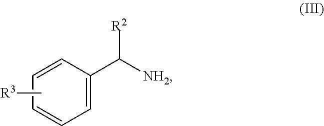

in which
R² is H, (C₁-C₄) alkyl, unsubstituted or substituted phenyl,
R³ is H, halogen, (C₁-C₄) alkyl, (C₁-C₄) alkoxy,
in the presence of H₂ and a catalyst in a solvent to form compounds of the general formula (IV) or salts thereof

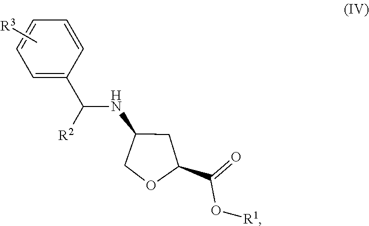

where R¹, R² and R³ are as defined above,
and said compounds are then converted in the presence of H₂ and a catalyst in a solvent into compounds of the general formula (I) or salts thereof.

Preferred definitions of the radicals for the compounds of the general formulae (I), (II), (III) and (IV) or salts thereof are as follows:
R¹ is (C₁-C₆) alkyl,
R² is H, phenyl, methyl,
R³ is H, F in para position of the phenyl ring, methyl-O- in para position of the phenyl ring, methyl in ortho position of the phenyl ring.

Particularly preferred definitions of the radicals for the compounds of the general formulae (I), (II), (III) and (IV) or salts thereof are as follows:
R¹ is CH₃,
R² is H,
R³ is H or methyl in ortho position of the phenyl ring.

The reaction sequence for preparing compounds of formula (I) or salts thereof is shown in Scheme 1.

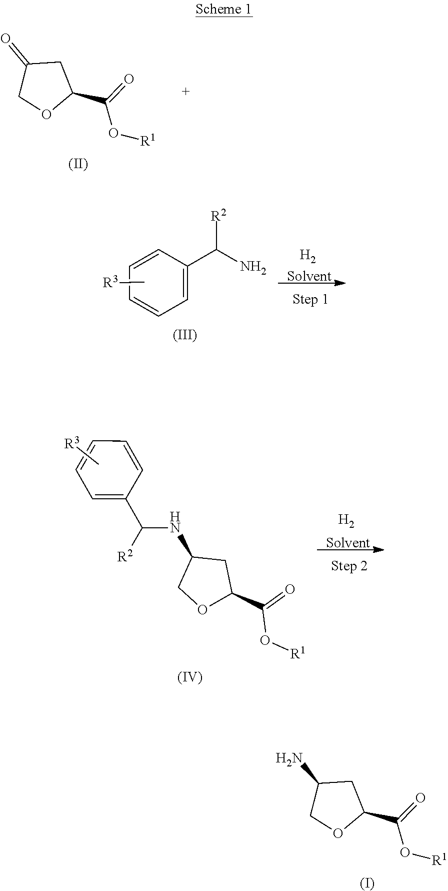

In the first reaction step, the compounds of the general formula (II) react with compounds of the general formula (III) or salts thereof (which may also be mixtures of the compounds of the general formula (III) and salts thereof) in the presence of hydrogen and a catalyst in a solvent to form compounds of the general formula (IV) or salts thereof.

The amines obtained here can be converted in a further step into cis-4-aminotetrahydrofuran-2-carboxylic esters of the general formula (I) or salts thereof using a catalyst and hydrogen in a solvent.

Both steps may also be combined in a one-stage process using a catalyst or a mixture of catalysts and hydrogen.

### Step 1:

The compounds of the general formulae (III) and (IV) may be present as free amines or in the form of their salts when acids are used; the salt form is preferred. Preferably, use is made of the following acids for salt formation with compounds of the general formula (II) and/or as additive: HCl, H₂SO₄, MsOH, TfOH, TFA. Particular preference is given to HCl.

The pressure during the reaction is 1 to 100 bar, preferably 1 to 50 bar, particularly preferably 20 to 50 bar.

The temperature during the reaction is 0° C. to 100° C., preferably 4° C. to 80° C., particularly preferably 20° C. to 40° C.

Catalysts used may be Raney nickel, Raney cobalt, Pt/C and Pt/Alox, preferably Raney nickel and Pt/C, particularly preferably Pt/C.

The catalysts are used in an amount of 0.1-10% by weight based on the compounds of the general formula (II); preferably, 0.5-5% by weight is used. The amount of catalysts used is calculated on the basis of the dry mass of the catalysts. The catalysts may be used dry or water-moist.

Lewis acids may additionally be added to the reaction mixture, such as: ZnCl₂, LiCl, MgCl₂, CeCl₃, CoCl₂, AlCl₃, FeCl₃, CaCl₂, Ti(iOPr)₄, B(OMe)₃; preferably, the reaction is conducted without Lewis acid additive.

Examples of suitable solvents and solvent mixtures are: R¹OH, R¹OH/toluene, 2-PrOH, 2-BuOH, t-amyl alcohol; preference is given to R¹OH and R¹OH/toluene.

The molar ratio of the compounds of the general formula (II) and (III) is in the range from about 0.5 to 2, preferably 0.9 to 1.1, particularly preferably 1.

If the compounds of the general formula (I) are obtained in the form of their salts, for example as a hydrochloride, the salt-free form can be obtained by treating the salt with a base, for example triethylamine.

### Step 2:

The compounds of the general formulae (I) and (IV) may be present as free amines or in the form of their salts with acids; the salt form is preferred. Preferably, use is made of the following acids for salt formation with compounds of formula (II) and/or as additive: HCl, H₂SO₄, MsOH, TfOH, TFA. Particular preference is given to HCl.

The pressure during the reaction is 1 to 100 bar, preferably 1 to 20 bar, particularly preferably 1 to 5 bar.

The temperature during the reaction is 0° C. to 100° C., preferably 20° C. to 80° C., particularly preferably 40° C. to 80° C.

Catalysts used may be Pd/C and Pd/Alox; preferably: Pd/C.

The catalysts are used in an amount of 0.1-10% by weight based on the compounds of formula (II); preferably, 0.1-2% by weight is used. The amount of catalysts used is calculated on the basis of the dry mass of the catalysts. The catalysts may be used dry or water-moist. Water-moist catalysts may be washed before the reaction with dry solvent under an inert gas atmosphere in order to remove adhering water. Alternatively, the water may be removed by azeotropic distillation with a suitable solvent. For example, toluene may be used for the azeotropic distillation.

The catalyst may be reused or used once or else preferably multiple times. Reactivation of the catalyst, for example by suitable washing, may be advantageous for the reuse thereof, for example by washing with methanol or acidic methanol.

Examples of suitable solvents and solvent mixtures are: R¹OH, R¹OH/toluene; preference is given to MeOH and MeOH/toluene.

If the compounds of the general formula (I) are obtained in the form of their salts, for example as a hydrochloride, the salt-free form can be obtained by treating the salt with a base, for example triethylamine.

It has now surprisingly been found that 4-oxotetrahydrofuran-2-carboxylic esters can be reacted directly in a reductive amination with optionally substituted phenylmethylamines of the general formula (III) or salts thereof in high cis selectivity and high yield using hydrogen and a catalyst in a solvent to give the corresponding N-substituted cis-4-aminotetrahydrofuran-2-carboxylic esters of the general formula (IV).

Since the different substitution pattern means that no conjugation and stabilization of the enamine by the ester as in WO 2012/137982 can take place, this was not obvious. By way of example, it was hitherto not possible to isolate a corresponding enamine from methyl 4-oxotetrahydrofuran-2-carboxylate analogously to the manner in WO 2012/137982. Instead, it is mainly the benzylamide of the ester that is formed under these conditions. Furthermore, reduction with hydrogen over a catalyst is necessary in order to obtain the desired cis selectivity. Reductive amination with ammonia or ammonium salts was also not successful.

### Elucidation of the Processes and Intermediates

### EXAMPLES

The present invention is elucidated in more detail by the examples which follow, without restricting the invention thereto.

### Measurement Methods

The products were characterized by ¹H NMR spectroscopy, standard HPLC from Agilent and/or GC-MS (Gas Chromatography Mass Spectrometry).

The NMR spectra were measured using a Bruker AV III 600.

The GC-MS samples were measured using a Shimadzu GCMS-QP-2010-Ultra coupled to an additional FID (flame ionization detector). To this end, the samples were first evaporated and then 1-10 mg of dry sample was admixed with 250 μl of N-methyl-N-trimethylsilyltrifluoroacetamide for silylation. After a reaction time of 1-5 minutes, the samples were diluted with 1 ml of acetonitrile and measured.

### Step 1 According to the Invention

### Example 1

### rac-cis-Methyl 4-(benzylamino)tetrahydrofuran-2-carboxylate hydrochloride

50 g (343 mmol, 1 eq) of methyl 4-oxotetrahydrofuran-2-carboxylate and 5 g (0.44 mmol) of 5% Pt/C catalyst (66% water-moist) were added to a solution of 49.3 g (343 mmol) of benzylamine hydrochloride and 250 g of methanol in a 600 ml autoclave. The autoclave was purged three times with 5 bar of argon and then the mixture was stirred under 5 bar of hydrogen for 16 h at 20° C. and 600 rpm. Thereafter, the autoclave was decompressed, the reaction mixture was filtered with suction through a suction filter and the residue was washed with methanol. The filtrate was concentrated to 290 g under reduced pressure and admixed with 250 g of cyclopentyl methyl ether (CPME). The solution was concentrated to 274 g under reduced pressure. The suspension formed was admixed with 50 g of CPME and 15 g of methanol, then stirred for 15 min at 40° C. and for 1 h at room temperature and then filtered with suction through a suction filter. The residue was washed with CPME/MeOH and dried under reduced pressure.

GC-MS (m/z): 205 [M-CH₂O], 190, 176, 146, 132, 114, 106, 91, 82, 65.

¹H NMR (DMSO-d6, 600 MHz): 2.1-2.2 (1H, m), 2.63-2.72 (1H, m), 3.69 (3H, s), 3.79-3.9 (1H, m), 3.9-4.05 (2H, m), 4.07-4.21 (2H, m), 4.47-4.55 (1H, t), 7.35-7.5 (3H, m), 7.51-7.61 (2H, m), 9.5-9.7 (2H, br) ppm.

Purity (qNMR): 94%

Yield: 56.4 g of the title compound (57% of theory)

### Examples 2-13

The following experiments have been carried out analogously to the experimental procedure for the preparation of Example 1. The yield of the target compound in solution was determined by quantitative NMR spectroscopy of the solution. The product was not isolated here.

| | Compound | Catalyst | | | | | | Yield, |
|---|---|---|---|---|---|---|---|---|
| | Formula | [% by | | Pressure | Temperature | Time | cis/trans | solution |
| No. | (III) | wt.] | Solvent | [bar] | [° C.] | [h] | ratio | [%] |
| 2 | BnNH₂ | 10 | MeOH | 5 | 20 | 16 | 7.1 | 59 |
| 3 | BnNH₂ | 10 | 2-PrOH | 5 | 20 | 16 | 7.3 | 51 |
| 4 | BnNH₂ | 10 | 2-BuOH | 5 | 20 | 16 | 5.9 | 72 |
| 5 | BnNH₂ | 3.5 | tert-amyl alcohol | 5 | 22 | 16 | 7.8 | 78 |
| 6* | BnNH₂ | 3.0 | MeOH | 5 | 22 | 16 | 5.4 | 76 |
| 7 | BnNH₂*HCl | 10 | MeOH | 50 | 20 | 16 | 6.6 | 80 |
| 8 | BnNH₂*HCl | 10 | MeOH | 30 | 35 | 16 | 6.6 | 77 |
| 9 | BnNH₂*HCl | 10 | MeOH/toluene | 5 | 20 | 16 | 7.3 | 80 |
| | | | (70/30) | | | | | |
| 10 | BnNH₂*HCl | 5 | MeOH/toluene | 20 | 60 | 16 | 6.6 | 66 |
| | | | (70/30) | | | | | |
| 11** | BnNH₂*HCl | 10 | MeOH/toluene | 5 | 20 | 16 | 9.8 | 65 |
| | | | (70/30) | | | | | |
| 12 | BnNH₂*HCl + | 3 | MeOH | 30 | 35 | 24 | 5.8 | 75 |
| | BnNH₂ | | | | | | | |
| 13*** | BnNH₂*HCl | 3 | MeOH | 30 | 35 | 24 | 5.0 | 76 |

*BnNH₂ metered over 4 h;
**10% by wt. of zinc chloride added;
***Compound (II) metered over 4 h.

### Example 14

### rac-cis-Methyl 4-(2-methylbenzylamino)tetrahydrofuran-2-carboxylate hydrochloride

50 g (343 mmol, 1 eq) of methyl 4-oxotetrahydrofuran-2-carboxylate and 5 g (0.44 mmol) of 5% Pt/C catalyst (66% water-moist) were added to a solution of 54.1 g (343 mmol) of 2-methylbenzylamine hydrochloride and 300 ml of methanol in a 600 ml autoclave. The autoclave was purged three times with 5 bar of argon and then the mixture was stirred under 5 bar of hydrogen for 16 h at 20° C. and 600 rpm. Subsequently, a further 2.5 g (0.22 mmol) of 5% Pt/C catalyst (66% water-moist) was added and further hydrogenation was performed under the same conditions. Thereafter, the autoclave was decompressed, the reaction mixture was filtered with suction through a suction filter and the residue was washed with methanol. The filtrate was concentrated to 125.7 g under reduced pressure.

GC-MS (m/z): 219 [M-CH₂O], 204, 190, 160, 146, 132, 120, 105, 91, 82, 79, 77, 69.

¹H NMR (DMSO-d6, 600 MHz): 2.19-2.28 (1H, m), 2.4 (3H, s), 2.7-2.78 (1H, m), 3.68 (3H, s), 3.9-4.02 (2H, m), 4.03-4.2 (3H, m), 4.47-4.55 (1H, t), 7.22-7.35 (3H, m), 7.55 (1H, d), 9.6-9.8 (2H, br) ppm.

Purity (qNMR): 57%

Yield: 125.7 g of the title compound (73% of theory)

### Step 2 According to the Invention

### Example 15

### rac-cis-Methyl 4-aminotetrahydrofuran-2-carboxylate hydrochloride

80 mg (0.025 mmol) of 5% Pd/C catalyst (33% water-moist) was added to a solution of 16 g (57.9 mmol) of cis-methyl 4-(benzylamino)tetrahydrofuran-2-carboxylate hydrochloride, 56 g of methanol and 24 g of toluene in a 300 ml autoclave. The autoclave was purged three times with 5 bar of argon and then the mixture was stirred under 5 bar of hydrogen for 16 h at 65° C. and 600 rpm. The autoclave was cooled to room temperature and decompressed. The reaction mixture was filtered with suction through a suction filter and the filtrate was concentrated under reduced pressure and then the solvent mixture was adjusted to a toluene:MeOH ratio of 90:10. The solution was cooled while stirring from 66° C. to room temperature and the suspension was filtered with suction through a suction filter at room temperature. The residue was washed with 5 ml each of toluene:MeOH=10:1 and toluene and then dried under reduced pressure.

GC-MS (m/z): 217 [M+TMS], 202, 187, 172, 158, 142, 128, 116, 100, 89, 73, 59, 54.

¹H NMR (DMSO-d6, 600 MHz): 1.95-2.04 (1H, m), 2.59-2.67 (1H, m), 3.70 (3H, s), 3.75-3.85 (2H, m), 3.9-3.97 (1H, m), 4.45-4.52 (1H, t), 8.2-8.5 (3H, br) ppm.

Purity (qNMR): 98%

Yield: 9.86 g of the title compound (92% of theory)

### Example 16

### rac-cis-Methyl 4-aminotetrahydrofuran-2-carboxylate hydrochloride

2 g (0.94 mmol) of 5% Pd/C catalyst was added to a solution of 125 g (56.9% purity, 249 mmol) of methyl cis-4-(2-methylbenzylamino)tetrahydrofuran-2-carboxylate hydrochloride and 250 g of methanol in a 600 ml autoclave. The autoclave was purged three times with 5 bar of argon and then the mixture was stirred under 5 bar of hydrogen for 16 h at 65° C. and 600 rpm. The autoclave was cooled to room temperature and decompressed. The reaction mixture was filtered with suction through a suction filter, the residue was washed with methanol and the filtrate was concentrated under reduced pressure. The residue was recrystallized with 22 g of methanol and 182 g of xylene. The crystal slurry was isolated by suction filtration using a suction filter, washed with xylene and dried under reduced pressure.

GC-MS (m/z): 217 [M+TMS], 202, 187, 172, 158, 142, 128, 116, 100, 89, 73, 59, 54.

¹H NMR (DMSO-d6, 600 MHz): 1.95-2.04 (1H, m), 2.59-2.67 (1H, m), 3.70 (3H, s), 3.75-3.85 (2H, m), 3.9-3.97 (1H, m), 4.45-4.52 (1H, t), 8.2-8.5 (3H, br) ppm.

Purity (qNMR): 80%

Yield: 52.4 g of the title compound (93% of theory)

1. A process for preparing compounds of general formula (I) or salts thereof 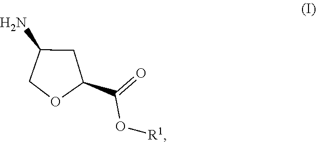
in which
R¹ is (C₁-C₆) alkyl or (C₃-C₆) cycloalkyl,
characterized in that in a first reaction step compounds of general formula (II) 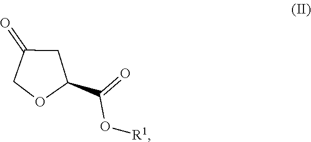
in which
R¹ is as defined above,
react with an amine of general formula (III) or salts thereof 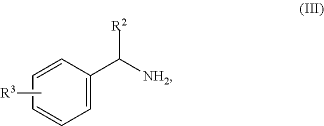
in which
R² is H, (C₁-C₄) alkyl, or an unsubstituted or substituted phenyl,
R³ is H, halogen, (C₁-C₄) alkyl or (C₁-C₄) alkoxy,
in the presence of H₂ and a catalyst in a solvent to form compounds of general formula (IV) or salts thereof 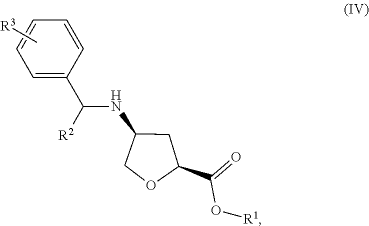
where R¹, R² and R³ are as defined above,
and said compounds are then converted in a second reaction step in the presence of H₂ and a catalyst in a solvent into compounds of the general formula (I) or salts thereof.

2. The process according to claim 1, characterized in that the definitions of the radicals for the compounds of the general formulae (I), (II), (III) and (IV) or salts thereof are as follows:
R¹ is (C₁-C₆) alkyl,
R² is H, phenyl, or methyl,
R³ is H, F in para position of the phenyl ring, methyl-O- in para position of the phenyl ring, or methyl in ortho position of the phenyl ring.

3. The process according to claim 2, characterized in that the definitions of the radicals for the compounds of the general formulae (I), (II), (III) and (IV) or salts thereof are as follows:
R¹ is CH₃,
R² is H,
R³ is H or methyl in ortho position of the phenyl ring.

4. The process according to claim 1, characterized in that the first reaction step is conducted at 20° C. to 40° C.

5. The process according to claim 1, characterized in that the second reaction step is conducted at 40° C. to 80° C.

6. The process according to claim 1, characterized in that the compounds of the general formulae (III), (IV) and (I) are present as hydrochloride salt.

7. The process according to claim 1, characterized in that the catalyst in the first reaction step is Pt/C.

8. The process according to claim 1, characterized in that the catalyst in the second reaction step is Pd/C.

9. The process according to claim 1, characterized in that the solvent is R¹OH, R¹OH/toluene, 2-PrOH, 2-BuOH or t-amyl alcohol.
